(19) United States
(12) Patent Application Publication
(10) Pub. No.: US 2019/0262799 A1
(43) Pub. Date: Aug. 29, 2019

(54) Mg-doped ALUMINA AEROGEL AND MANUFACTURING METHOD THEREOF

(71) Applicant: Chung Yuan Christian University, Taoyuan City (TW)

(72) Inventors: Yi-Feng Lin, Taoyuan City (TW); Yong-Jie Lin, New Taipei City (TW)

(73) Assignee: Chung Yuan Christian University, Taoyuan City (TW)

(21) Appl. No.: 15/968,761

(22) Filed: May 2, 2018

(30) Foreign Application Priority Data
Feb. 26, 2018 (TW) 107106321

(51) Int. Cl. B01J 23/02; B01J 35/00; B01J 37/03; B01J 37/02; C01F 7/16; C07D 317/38

(52) U.S. Cl. CPC B01J 23/02; B01J 35/0013; B01J 37/036; C01P 2002/30; C01F 7/162; C07D 317/38; C01P 2002/54; B01J 37/0236

(57) ABSTRACT

A Mg-doped alumina aerogel and a manufacturing method thereof are provided. The Mg-doped alumina aerogel is a three-dimensional cross-linked network structure including a plurality of magnesium atoms, a plurality of aluminum atoms, a plurality of oxygen atoms, and a plurality of hydrogen atoms. The three-dimensional cross-linked network structure has a —Mg—O—Al— bond at least on the main chain.

### CROSS-REFERENCE TO RELATED APPLICATION

This application claims the priority benefit of Taiwan application serial no. 107106321, filed on Feb. 26, 2018. The entirety of the above-mentioned patent application is hereby incorporated by reference herein and made a part of this specification.

### BACKGROUND OF THE INVENTION

### Field of the Invention

The invention relates to an aerogel and a manufacturing method thereof, and more particularly, to a Mg-doped alumina aerogel and a manufacturing method thereof.

### Description of Related Art

Carbon dioxide has always been the main greenhouse gas most needed to be addressed in the atmosphere. In recent years, carbon dioxide capture and storage (CCS) is still the main emission reduction technique, but storing captured carbon dioxide raises safety concerns, and therefore many researchers aim to reuse carbon dioxide to effectively reduce carbon dioxide in the atmosphere.

In the past, significant effort has been put into synthesizing cyclic carbonate using epoxide and carbon dioxide. Cyclic carbonate is an engineering plastic having numerous advantages such as excellent heat resistance, pressure resistance, good moldability, and transparency. In the reaction of epoxide and carbon dioxide to prepare cyclic carbonate, a catalyst is needed to catalyze the cycloaddition reaction (such as formula (I) below, wherein R is an alkyl group). A common catalyst is, for instance, ionic liquid, alkali metal salt, and metal oxide. However, ionic liquid is a homogeneous catalyst and is not easily separated; the alkali metal salt needs a solvent having a hydrogen bond, and the alkali metal salt is dissolved in cyclopropane such that it is not easily to be separated. Moreover, metal oxide requires a solvent harmful to the environment or damaging to a heterogeneous catalyst or co-catalyst, such as dimethylformamide (DMF). In particular, DMF is highly toxic, is harmful to the environment, expensive, and the product is not easily separated.

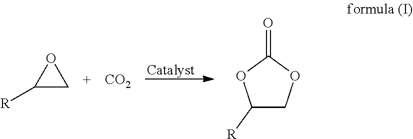

Based on the above, how to develop a catalyst that is easily separated, environmentally friendly, lower cost, and has high catalytic efficiency is an important topic requiring research.

### SUMMARY OF THE INVENTION

The invention provides a Mg-doped alumina aerogel having the advantages of easy separation, environmental friendliness, lower cost, and high catalytic efficiency and can be used as a catalyst for preparing cyclic carbonate.

The invention provides a Mg-doped alumina aerogel that is a three-dimensional cross-linked network structure including a plurality of magnesium atoms, a plurality of aluminum atoms, a plurality of oxygen atoms, and a plurality of hydrogen atoms. The three-dimensional cross-linked network structure has a —Mg—O—Al— bond at least on the main chain.

In an embodiment of the invention, the molar ratio of the plurality of aluminum atoms and the plurality of magnesium atoms is 5:5 to 9:1.

In an embodiment of the invention, the molar ratio of the plurality of aluminum atoms and the plurality of magnesium atoms is 6:4 to 8:2.

In an embodiment of the invention, the three-dimensional cross-linked network structure further has an amine group.

The invention further provides a manufacturing method of a Mg-doped alumina aerogel including performing a hydrolysis condensation reaction on an aluminum salt and a magnesium salt in the presence of an epoxide to form a gel; and performing a drying treatment on the gel to form the Mg-doped alumina aerogel.

In an embodiment of the invention, the manufacturing method of the Mg-doped alumina aerogel further includes an amine group modification step, in which a mixture of a silane compound containing an amine group and an alcohol solvent is added in the gel.

In an embodiment of the invention, the amine group modification step is performed one to three times.

In an embodiment of the invention, the aluminum salt is aluminum nitrate.

In an embodiment of the invention, the magnesium salt is magnesium nitrate.

In an embodiment of the invention, the drying treatment is a supercritical fluid drying method.

Based on the above, the invention provides a Mg-doped alumina aerogel including a three-dimensional cross-linked network structure. The three-dimensional cross-linked network structure has a —Mg—O—Al— bond at least one the main chain, wherein the magnesium atom has Lewis base activity and the aluminum atom has Lewis acid activity, and therefore the three-dimensional cross-linked network structure can be used as a catalyst, and the three-dimensional cross-linked network structure has the advantages of easy separation and environmental friendliness. It should be mentioned that, when the Mg-doped alumina aerogel is applied in a catalyst in the cycloaddition reaction of epoxide and carbon dioxide, high catalytic efficiency is achieved without a co-catalyst and a solvent. Moreover, the invention further provides a Mg-doped alumina aerogel modified by an amine group. In addition to having the advantages of easy separation and environmental friendliness, the Mg-doped alumina aerogel modified by the amine group also has better thermal stability, and since the selectivity of the catalytic reaction is higher, better catalytic efficiency is achieved.

In order to make the aforementioned features and advantages of the disclosure more comprehensible, embodiments accompanied with figures are described in detail below.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1 is a catalytic cycle diagram of a Mg-doped alumina aerogel modified by an amine group applied in the preparation of cyclic carbonate using epoxide and carbon dioxide according to an embodiment of the invention.

FIG. 2A is a field-emission scanning electron microscope image of experimental example 1.

FIG. 2B is a field-emission scanning electron microscope image of experimental example 2.

FIG. 2C is a field-emission scanning electron microscope image of experimental example 3.

FIG. 2D is a field-emission scanning electron microscope image of experimental example 4.

FIG. 2E is a field-emission scanning electron microscope image of experimental example 5.

FIG. 2F is a field-emission scanning electron microscope image of comparative example 1.

FIG. 3A is nitrogen adsorption and desorption curve of experimental examples 1 to 5 and comparative example 1.

FIG. 3B is pore size distribution curve of experimental examples 1 to 5 and comparative example 1.

FIG. 4 is a thermogravimetric analysis curve of experimental example 2.

FIG. 5A is a field-emission scanning electron microscope image of experimental example 6.

FIG. 5B is a field-emission scanning electron microscope image of experimental example 7.

FIG. 5C is a field-emission scanning electron microscope image of experimental example 8.

FIG. 6 is an infrared spectrum of experimental example 8.

FIG. 7 is a thermogravimetric analysis curve of experimental example 8.

### DESCRIPTION OF THE EMBODIMENTS

### First Example

The Mg-doped alumina aerogel of the first embodiment is a three-dimensional cross-linked network structure having the features of low density, continuous pores, high porosity, and high specific surface area, and is suitable as a catalyst. The three-dimensional cross-linked network structure of the Mg-doped alumina aerogel includes a plurality of magnesium atoms, a plurality of aluminum atoms, a plurality of oxygen atoms, and a plurality of hydrogen atoms. The three-dimensional cross-linked network structure has a —Mg—O—Al— bond formed by one of a plurality of magnesium atoms, one of a plurality of oxygen atoms, and one of a plurality of aluminum atoms at least one the main chain; and a hydroxyl group formed by one of a plurality of oxygen atoms and one of a plurality of hydrogen atoms. In particular, the magnesium atom has Lewis base activity and the aluminum atom has Lewis acid activity, and therefore the Mg-doped alumina aerogel can be used as the catalyst. Moreover, when the Mg-doped alumina aerogel is used as the catalyst, the advantages of easy separation, environmental friendliness, and high catalytic efficiency are achieved.

Specifically, the bonding method of the three-dimensional cross-linked network structure of the first embodiment is, for instance, the bonding method represented by formula (II).

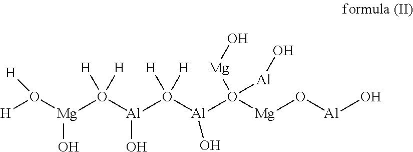

In formula (II), the oxygen atom is bonded to at least one of an aluminum atom and a magnesium atom. When one of the bonds of the oxygen atom is connected to an aluminum atom or a magnesium atom, another bond can be connected to a hydrogen atom, and the lone pair of electrons of the oxygen atom can be further bonded to other hydrogen atoms. When the two bonds of the oxygen atom are connected to an aluminum atom at the same time, connected to a magnesium atom at the same time, or respectively connected to a magnesium atom and an aluminum atom, the lone pair of electrons of the oxygen atom can be bonded to hydrogen atoms, other magnesium atoms, or other aluminum atoms.

In the three-dimensional cross-linked network structure, the molar ratio of a plurality of aluminum atoms and a plurality of magnesium atoms can be 5:5 to 9:1, preferably 6:4 to 8:2, and more preferably 7:3 to 8:2. When the molar ratio of the plurality of aluminum atoms and the plurality of magnesium atoms is 6:4 to 8:2, the catalytic efficiency of using the Mg-doped alumina aerogel as the catalyst can be further enhanced. When the molar ratio of a plurality of aluminum atoms and a plurality of magnesium atoms is 7:3 to 8:2, the structural stability of the Mg-doped alumina aerogel can be further increased.

By measuring via a Brunauer-Emmett-Teller (BET) analyzer, the BET specific surface area of the Mg-doped alumina aerogel can be 37 m²/g to 470 m²/g, preferably 130 m²/g to 465 m²/g; and the average pore diameter of the Mg-doped alumina aerogel can be 2 nm to 50 nm.

### Second Example

The three-dimensional cross-linked network structure of the Mg-doped alumina aerogel of the second example is substantially similar to the three-dimensional cross-linked network structure of the Mg-doped alumina aerogel of the first example, but the side chain of the three-dimensional cross-linked network structure of the second example further has an amine group. In an embodiment, the amine group is bonded to one of a plurality of magnesium atoms and/or one of a plurality of aluminum atoms via a —Y—Si—O— bond, and Y is a C1 to C10 alkylene group. In particular, the amine group has Lewis base activity, and can be used as an active site of a catalytic reaction with the magnesium atom and the aluminum atom together to further increase the selectivity of the catalytic reaction and increase the catalytic efficiency. It should be mentioned that, by modifying the amine group on the three-dimensional cross-linked network structure of the Mg-doped alumina aerogel, the hydroxyl group on the three-dimensional cross-linked network structure can be reduced, such that the three-dimensional cross-linked network structure of the Mg-doped alumina aerogel is more stable and the thermal stability thereof is better. Moreover, when the Mg-doped alumina aerogel modified by an amine group is applied in a catalytic reaction, the selectivity of the catalytic reaction can be higher to achieve better catalytic efficiency.

Specifically, the bonding method of the three-dimensional cross-linked network structure of the second embodiment is, for instance, the bonding method represented by formula (III). Formula (III) is similar to formula (II), but in formula (III), an amine group is further present on a side chain, and the amine group is bonded to a magnesium atom and an aluminum atom via a —(CH₂)₃—Si—O— bond.

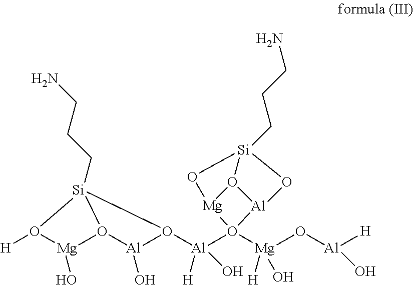

### [Manufacturing Method of Mg-Doped Alumina Aerogel of First Example]

The manufacturing method of the Mg-doped alumina aerogel of the first embodiment includes a (a) gel-forming step and a (b) drying treatment.

(a) Gel-forming step: the method for forming a gel is an epoxide-initiated gelation including performing a hydrolysis condensation reaction on aluminum salt and magnesium salt used as precursors of a metal oxide in the presence of an epoxide and a solvent to form a gel. Specifically, aluminum salt and magnesium salt are used as precursors, and are dissolved in a solvent to form hydrated aluminum ion Al(H₂O)₆³⁺ and hydrated magnesium ion Mg(H₂O)₆²⁺. Next, an epoxide is added as a proton-removing agent. The hydrated aluminum ion and the hydrated magnesium ion form Al(OH)(H₂O)₅²⁺ and Mg(OH)(H₂O)₅⁺ due to loss of protons, and the ionic groups continue to be polymerized to form ion clusters with small molecular weight, and are reacted in a condensation reaction with water molecules in the solvent to form ion clusters such as [(H₂O)₅AlOAl(H₂O)₅]⁴⁺, [(H₂O)₅AlOMg(H₂O)₅]³⁺, and [(H₂O)₅MgOMg(H₂O)₅]²⁺. These ions are cross-linked repeatedly to form a three-dimensional cross-linked network structure having a —Mg—O—Al— bond.

In an embodiment, 1,2-propylene oxide is used as epoxide, hydrated aluminum nitrate is used as aluminum salt, and hydrated magnesium nitrate is used as magnesium salt as an example, and the detailed reaction mechanism is shown in formula (IV-1) to formula (IV-6).

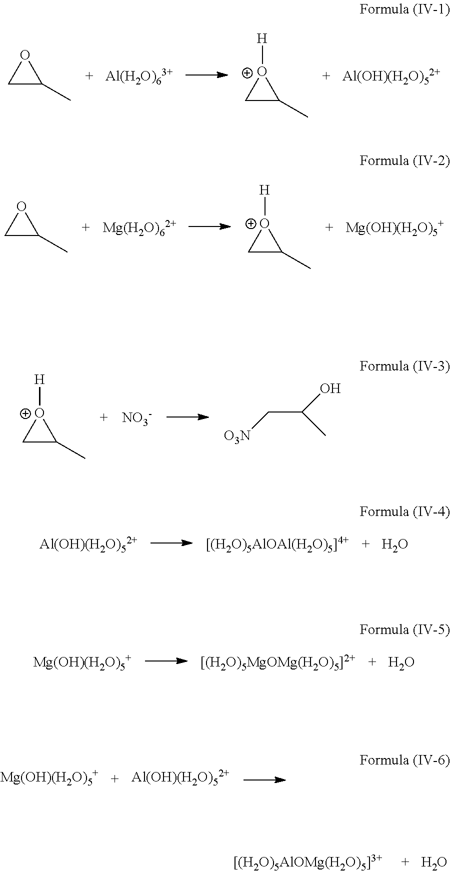

The epoxide is not particularly limited as long as the protons of hydrated aluminum ion Al(H₂O)₆³⁺ and hydrated magnesium ion can be captured. The epoxide is a compound having an epoxy group or an oxetanyl group. Specifically, the epoxide includes ethylene oxide, 1,2-propylene oxide, 1,3-propylene oxide, 1,2-butylene oxide, 1,3-butylene oxide, 1,2-epoxypentane, 1,3-butylene oxide, 2,3-butylene oxide, and 1,3-butylene oxide. The epoxides can be used alone or used in a combination of two or more. The molar ratio of aluminum in the aluminum salt and magnesium in the magnesium salt and the epoxide ((Mg+Al)/epoxide) can be 1:4 to 1:20, preferably 1:10 to 1:20. When the molar ratio is in the above range, the yield of the gel is better. If the amount of the epoxide is too small, then gelation is difficult. If the amount of the epoxide is too great, then reaction is incomplete, such that the epoxide undergoes self-polymerization and the pores collapse during drying as a result.

The solvent is not particularly limited as long as the solvent can provide oxygen and hydrogen. The solvent can be water, an alcohol solvent, or a ketone solvent. Specific examples of the alcohol solvent include, for instance, methanol, ethanol, n-propanol, isopropanol, n-butanol, sec-butanol, tert-butanol, isobutanol, n-hexanol, n-heptanol, n-octanol, and n-decanol. Specific examples of the ketone solvent include 1-octanone, 2-octanone, 1-nonanone, 2-nonanone, acetone, 2-heptanone (methyl amyl ketone), 4-heptanone, 1-hexanone, 2-hexanone, diisobutyl ketone, cyclohexanone, methyl cyclohexanone, phenyl acetone, methyl ethyl ketone, methyl isobutyl ketone, acetyl acetone, and acetonyl acetone. In terms of reactivity, the solvent is preferably water or an alcohol solvent. Moreover, in terms of gelling effect, the solvent is more preferably methanol. The solvent can be used alone or used in a combination of two or more. The amount of the solvent is not particularly limited as long as the solvent can sufficiently dissolve the magnesium salt, aluminum salt, and epoxide.

The aluminum salt is not particularly limited as long as the aluminum salt can produce aluminum ions. Specific examples of the aluminum salt include aluminum nitrate, aluminum sulfate, aluminum carbonate, aluminum oxide, aluminum hydroxide, and aluminum chloride. The aluminum salt can be used alone or used in a combination of two or more. Moreover, in terms of reactivity, the aluminum salt is preferably aluminum salt hydrate, such as hydrated aluminum nitrate.

The magnesium salt is not particularly limited as long as the aluminum salt can produce magnesium ions. Specific examples of the magnesium salt include magnesium nitrate, magnesium sulfate, magnesium carbonate, and magnesium chloride. The magnesium salt can be used alone or used in a combination of two or more. Moreover, in terms of reactivity, the magnesium salt is preferably magnesium salt hydrate, such as hydrated magnesium nitrate. Moreover, the magnesium salt and the aluminum salt preferably adopt the same anion. For instance, when magnesium nitrate is selected as the magnesium salt, aluminum nitrate is preferably selected as the aluminum salt.

The molar ratio of the aluminum in the aluminum salt and the magnesium in the magnesium salt can be 5:5 to 9:1, preferably 6:4 to 8:2, and more preferably 7:3 to 8:2. When the molar ratio of the aluminum in the aluminum salt and the magnesium in the magnesium salt is 6:4 to 8:2, the catalytic efficiency of using the Mg-doped alumina aerogel as the catalyst can be further enhanced. When the molar ratio of the aluminum in the aluminum salt and the magnesium in the magnesium salt is 7:3 to 8:2, the structural stability of the Mg-doped alumina aerogel can be further increased. Moreover, via an inductively-coupled plasma spectrometer, the inventors discovered that the molar ratio of aluminum of the aluminum salt and magnesium of the magnesium salt is the same as the molar ratio of aluminum atoms and magnesium atoms in the Mg-doped alumina aerogel, and therefore in the present application, the metal atom ratio in the Mg-doped alumina aerogel is successfully controlled via the metal atom ratio of the precursors.

The hydrolysis condensation reaction of the aluminum salt and the magnesium salt is preferably performed in an alkaline environment. To provide an entirely alkaline environment for the solution, an alkaline compound can be added. The alkaline compound is not particularly limited as long as the alkaline compound can adjust the solution to alkaline. The alkaline compound includes ammonium hydroxide, tetramethylammonium hydroxide, tetraethylammonium hydroxide, sodium hydroxide, potassium hydroxide, magnesium hydroxide, calcium hydroxide, and barium hydroxide. The alkaline compound can be used alone or used in a combination of two or more. The molar ratio of aluminum in the aluminum salt and magnesium in the magnesium salt and the alkaline compound ((Mg+Al)/alkali) can be 1:0.5 to 1:2, preferably 1:1. When the molar ratio is in the above range, the yield of the gel is better.

In the (a) gel-forming step, the order of addition of the reagent is not particularly limited. However, in terms of reactivity, the aluminum salt and the magnesium salt are preferably added in the solvent first to sufficiently dissolve the aluminum salt and the magnesium salt in the solvent, and then ammonium hydroxide is added to adjust the pH value. Lastly, after epoxide is added and the components are evenly mixed, the mixture is left to stand for 6 hours to 24 hours to stably form a gel. If the mixture is not left to stand long enough, then the gel is broken and gelling does not easily occur, and if the mixture is left to stand too long, then the gel dries automatically.

Moreover, in the (a) gel-forming step, an aging treatment (such as replacing the solvent) can be further performed to increase the stress of the gel against the subsequent drying treatment. Specifically, by replacing the solvent with a solvent having a smaller surface tension, the supercritical fluid more easily replaces the solvent in a subsequent drying treatment to maintain the porous structure of the gel. The time of the aging treatment is 24 hours. The aging treatment can be performed once or more.

(b) Drying treatment: after the drying treatment of the gel, the Mg-doped alumina aerogel (powder) can be formed. The drying treatment is preferably a supercritical fluid drying method including injecting a supercritical fluid in a gel at high temperature and high pressure to maintain pore integrity in the gel and replacing the solvent in the pores completely with the supercritical fluid, and then the environment is adjusted to atmospheric pressure and room temperature. The supercritical fluid at this point is changed back to a regular gas, and an aerogel is thus formed. An aerogel having many active points, low density, pore continuity, and high specific surface area can be obtained by drying using a supercritical fluid, and the aerogel is suitable for a catalytic reaction. The supercritical fluid is not particularly limited, and can be suitably selected based on need. The supercritical fluid is, for instance, carbon dioxide, methane, acetone, or propylene. In terms of safety and convenience, the supercritical fluid is preferably carbon dioxide. The time of the drying treatment can be 4 hours or more. If the time of the drying treatment is too short, then drying is incomplete.

### [Manufacturing Method of Mg-Doped Alumina Aerogel of Second Example]

The manufacturing method of the Mg-doped alumina aerogel of the second example includes performing a (c) amine group-modification step after the (a) gel-forming step. The object of the (c) amine group-modification step is to modify an amine group having Lewis base activity on a side chain of the Mg-doped alumina aerogel to increase active site and to reduce the hydroxyl group on the three-dimensional cross-linked network structure such that the three-dimensional cross-linked network structure of the Mg-doped alumina aerogel is more stable and the thermal stability thereof is better.

The (c) amine group-modification step includes adding a mixture of a silane compound containing an amine group and an alcohol solvent in the gel and leaving the mixture to stand for 24 hours. The silane compound containing the amine group is, for instance, the compound represented by formula (1).

(NH₂—Y)ₘ—Si(OR)₄₋ₘ formula (1)

In formula (1), Y is a C1 to C10 alkylene group, R is a C1 to C10 alkyl group, and m is an integer of 1 to 3, wherein any —CH₂— in the alkylene group can be replaced by —NH—.

Specific examples of the compound represented by formula (1) include N-(2-aminoethyl)-3-aminopropylmethyldimethoxysilane, N-(2-aminoethyl)-3-aminopropyltrimethoxysilane, 3-aminopropyl trimethoxysilane, and 3-aminopropyl triethoxysilane. In terms of accessibility and catalytic efficiency, the compound represented by formula (1) is preferably 3-aminopropyl trimethoxysilane.

In terms of reaction rate, the compound represented by formula (1) is preferably 3-aminopropyl trimethoxysilane. As shown in formula (V), a dehydration reaction can occur between the 3-aminopropyl trimethoxysilane and a hydroxyl group on the Mg-doped alumina aerogel surface, such that silicon is bonded to three oxygen atoms of a metal (magnesium or aluminum) so as to form a Mg-doped alumina aerogel having a surface modified by an amine group.

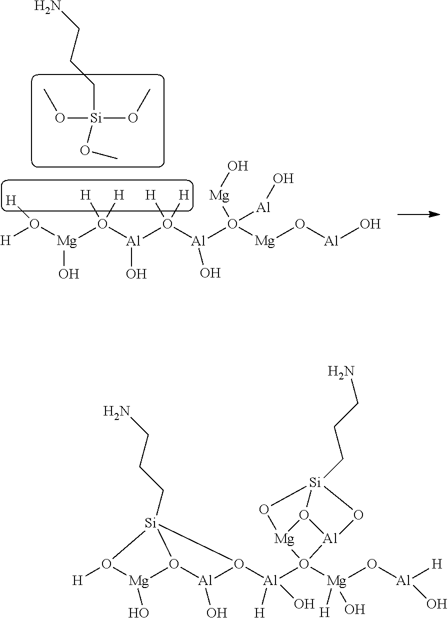

The amine group-modification step can be performed once or more, preferably one to three times, and more preferably three times. When the amine group-modification step is performed three times, the Lewis acid and the Lewis base in the Mg-doped alumina aerogel reach the optimal balance, such that the catalytic efficiency of the catalytic reaction is optimal. When the amine group-modification step is repeated more than three times, the surface is full of amine groups and the silane compound containing an amine group may undergo self-polymerization such that the active site of the Lewis base is covered. As a result, during a catalytic reaction, the reactants cannot be completely reacted at the active site due to steric effects.

Specific examples of the alcohol solvent used in the (c) amine group-modification step are the same as the specific examples of the alcohol solvent used in the (a) gel-forming step and are not repeated herein.

Based on a total weight of 100 wt % of the silane compound containing an amine group and an alcohol solvent, the content of the silane compound containing an amine group is 10 wt % to 30 wt %. If the content of the silane compound containing an amine group is insufficient, then the modification effect is poor; and if the content of the silane compound containing an amine group is too large, then the silane compound containing an amine group may undergo self-polymerization, such that the active site of the original Lewis acid and base is covered.

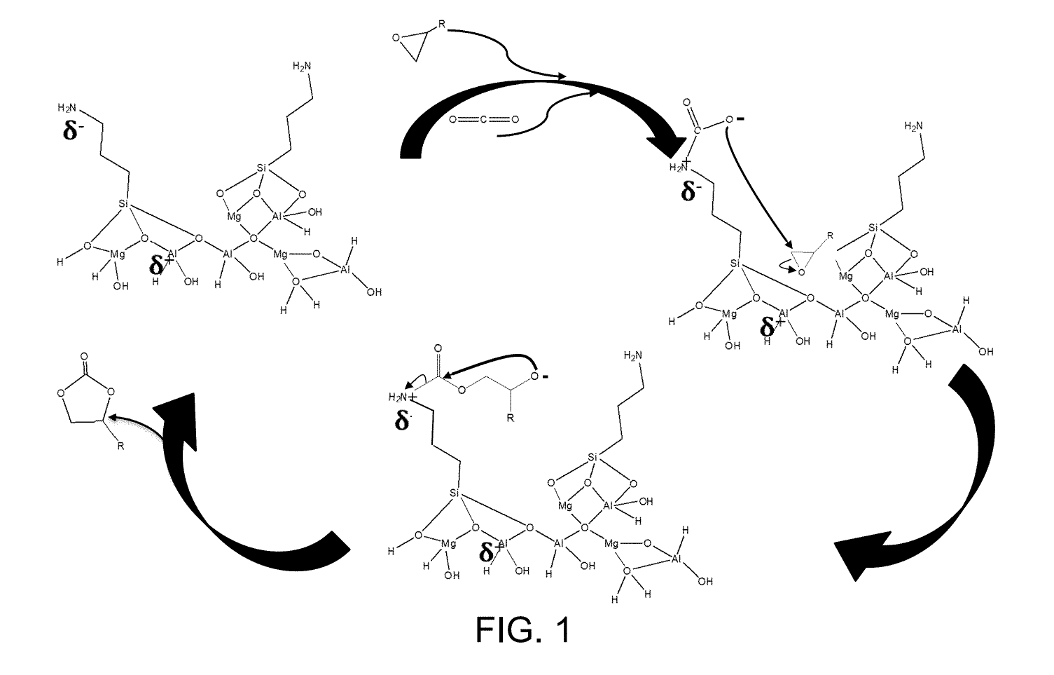

Moreover, the catalytic cycle diagram of using the Mg-doped alumina aerogel modified by an amine group as a catalyst for the preparation of cyclic carbonate using epoxide and carbon dioxide is shown in FIG. 1, wherein R on the epoxide is an alkyl group. In FIG. 1, the amine group on the three-dimensional cross-linked network structure can be used as a Lewis base such that the carbon dioxide adsorbed thereto becomes a nucleophile (acidic group is produced), and the aluminum on the three-dimensional cross-linked network structure can be used as a Lewis acid to adsorb epoxide. Next, an acidic group can perform a nucleophilic attack to open the ring of the epoxide. Lastly, the oxygen anion of the intermediate product can perform a nucleophilic attack on a carbonyl group and produce cyclic carbonate via a series of reactions. It should be mentioned that, the magnesium on the three-dimensional cross-linked network structure can also be used as a Lewis base to perform the catalytic reaction, and therefore when the Mg-doped alumina aerogel modified by an amine group is applied in a catalytic reaction, the active sites of two Lewis bases can be provided, such that the selectivity of the catalytic reaction is higher to achieve a better catalytic efficiency. Moreover, the mechanism in which the magnesium on the three-dimensional cross-linked network structure is used as a Lewis base to perform a catalytic reaction is similar to the mechanism in which an amine group is used as a Lewis base to perform a catalytic reaction and is not repeated herein.

The following experimental examples are used to further describe the invention. However, it should be understood that, the experimental examples are only exemplary, and are not intended to limit the implementation of the invention.

### Experimental Examples 1 to 5, Comparative Example 1

First, aluminum nitrate (Al(NO₃)₃.9H₂O, made by Alfa Aesar Corporation) and magnesium nitrate (Mg(NO₃)₂.6H₂O, made by Alfa Aesar Corporation) were added in 10 mL of methanol (used as solvent, made by Tedia Corporation) according to the molar ratio of the precursors of the metal oxides of experimental examples 1 to 5 and comparative example 1 shown in Table 1, and then stirring was performed until the solution cleared. Next, ammonia water (ammonium hydroxide, NH₄OH, made by Fisher Scientific, ammonium hydroxide concentration: 25% to 30%) was added and stirring was performed for 10 minutes, and then 1,2-propylene oxide (made by Alfa Aesar) was added and stirring was performed for 5 minutes to evenly mix the components, and then the mixture was left to stand to gel. After gelling, ethanol was used to replace the solvent for 24 hours to age the gel, wherein the molar ratio of the metal precursor:ammonia water:propylene oxide is 1:1:16. Next, the gel was removed and placed in a carbon dioxide supercritical drying stainless steel tank, and then heating was performed and a supercritical carbon dioxide fluid was introduced to increase the pressure. After drying at a high-temperature and high-pressure environment for 4.5 hours, the Mg-doped alumina aerogel of experimental examples 1 to 5 or the alumina aerogel of comparative example 1 were obtained.

### Experimental Examples 6 to 9

In experimental examples 6 to 9, the Mg-doped alumina aerogel modified by an amine group was respectively formed via the precursors of the meal oxides, solvents, alkali compounds, and epoxides of the same type and amount as experimental examples 1 to 5 and the same steps. However, the molar ratio of aluminum nitrate (Al(NO₃)₃.9H₂O) and magnesium nitrate (Mg(NO₃)₂.6H₂O) was fixed at 8:2, and after gelling and aging, an amine group-modification step was performed. The amine group-modification step can be performed by leaving to stand for 24 hours using ethanol containing 15 wt % of 3-aminopropyl trimethoxysilane (made by Acros Corporation). If a plurality of modifications is needed, then a plurality of amine group-modification steps needs to be performed.

### Experimental Example 10

In experimental example 10, an alumina aerogel was formed via the precursor of the metal oxide, solvent, alkali compound, and epoxide of the same type and amount as comparative example 1 and the same steps. However, after gelling and aging, the amino group-modification step was performed three times. The amine group-modification step of experimental example 10 is the same as in experimental examples 6 to 9 and is not repeated herein.

### <Evaluation Methods>

a. Field-emission scanning electron microscope (FESEM): the sample was attached to a platform via copper adhesive, and then a platinum conductive layer was coated, and an image was captured using a field-emission scanning electron microscope (model: S4800, made by Hitachi Corporation).

b. Specific surface area and pore size distribution analyzer: the specific surface area and pore size distribution were measured via a BET analyzer (surface area and porosity analyzer ASAP 2020, made by Micromeritics Co., Ltd.) and a nitrogen adsorption method.

c. Thermogravimetric analyzer: a thermogravimetric analysis curve was measured by heating until 100° C. at a heating rate of 20° C./min using a thermogravimetric analyzer (TGA) (model: Q50, made by Waters Corporation) in a nitrogen environment.

d. Fourier transform infrared spectroscopy (FT-IR): measured via a Fourier transform infrared spectroscope (model: TENSOR-27, made by Bruker Spectroscopy Co., Ltd.)

e. Catalytic reaction test: 20 mmol g of 1,2-propylene oxide, 0.2 g of a catalyst (i.e., aerogel of experimental examples 1 to 10 and comparative example 1), and a magnet were placed in an autoclave, and carbon dioxide was introduced (pressure: 10 kgw). Next, the autoclave was heated to a reaction temperature of 150° C. using a heating package to react for 15 hours. After the reaction was complete, the autoclave was placed in an ice bath to perform cooling, and after room temperature was achieved, unreacted carbon dioxide gas in the autoclave was slowly discharged. After degassing, the product was removed to perform an NMR proton analysis (model: Bruker Avance II 400 MHz, made by Bruker Spectroscopy Co., Ltd.) The test reagent of the NMR proton analysis is deuterated chloroform (CDCl₃), and the byproduct polypropylene oxide (PPO) is produced in the reaction process. The conversion rate, selectivity, and yield can be calculated from the analysis results of the NMR proton spectroscopy.

TABLE 1

| | | | | | | Pore characteristics | | Catalytic reaction test | | |
|---|---|---|---|---|---|---|---|---|---|---|
| | | | | | | Average | Specific | Propylene | Propylene | |
| | Al:Mg | Aluminum | Magnesium | Ammonium | Propylene | pore | surface | oxide | carbonate | Propylene |
| | (molar | nitrate | nitrate | hydroxide | oxide | size | area | conversion | selectivity | carbonate |
| | ratio) | (moles) | (moles) | (moles) | (moles) | (nm) | (m²/g) | rate (%) | (%) | yield (%) |
| Experimental | 9:1 | 0.9 | 0.1 | 1 | 16 | 3.6 | 465 | 97.06 | 42.18 | 40.94 |
| example 1 | | | | | | | | | | |
| Experimental | 8:2 | 0.8 | 0.2 | 1 | 16 | 4.8 | 364 | 95.01 | 59.89 | 56.90 |
| example 2 | | | | | | | | | | |
| Experimental | 7:3 | 0.7 | 0.3 | 1 | 16 | 2.7 | 221 | 98.23 | 57.44 | 56.42 |
| example 3 | | | | | | | | | | |
| Experimental | 6:4 | 0.6 | 0.4 | 1 | 16 | 5.9 | 130 | 95.20 | 51.39 | 47.53 |
| example 4 | | | | | | | | | | |
| Experimental | 5:5 | 0.5 | 0.5 | 1 | 16 | 13.6 | 37 | 73.16 | 55.74 | 41.89 |
| example 5 | | | | | | | | | | |
| Comparative | 10:0 | 1 | 0 | 1 | 16 | 3.2 | 450 | 97.42 | 25.69 | 25.03 |
| example 1 | | | | | | | | | | |

TABLE 2

| | | | | | | | Catalytic reaction test | | |
|---|---|---|---|---|---|---|---|---|---|
| | | | | | | | Propylene | Propylene | |
| | Al:Mg | Aluminum | Magnesium | Ammonium | Propylene | Times of | oxide | carbonate | Propylene |
| | (molar | nitrate | nitrate | hydroxide | oxide | amine group | conversion | selectivity | carbonate |
| | ratio) | (moles) | (moles) | (moles) | (moles) | modification | rate (%) | (%) | yield (%) |
| Experimental | 8:2 | 0.8 | 0.2 | 1 | 16 | 0 | 95.01 | 59.89 | 56.90 |
| example 2 | | | | | | | | | |
| Experimental | 8:2 | 0.8 | 0.2 | 1 | 16 | 1 | 98.04 | 76.06 | 74.57 |
| example 6 | | | | | | | | | |
| Experimental | 8:2 | 0.8 | 0.2 | 1 | 16 | 2 | 98.72 | 87.04 | 85.92 |
| example 7 | | | | | | | | | |
| Experimental | 8:2 | 0.8 | 0.2 | 1 | 16 | 3 | >99 | >99 | 99 |
| example 8 | | | | | | | | | |
| Experimental | 8:2 | 0.8 | 0.2 | 1 | 16 | 4 | 94.08 | 83.80 | 78.84 |
| example 9 | | | | | | | | | |
| Experimental | 10:0 | 1.0 | 0 | 1 | 16 | 3 | 97.74 | 83.31 | 81.4 |
| example 10 | | | | | | | | | |

### <Evaluation Results>

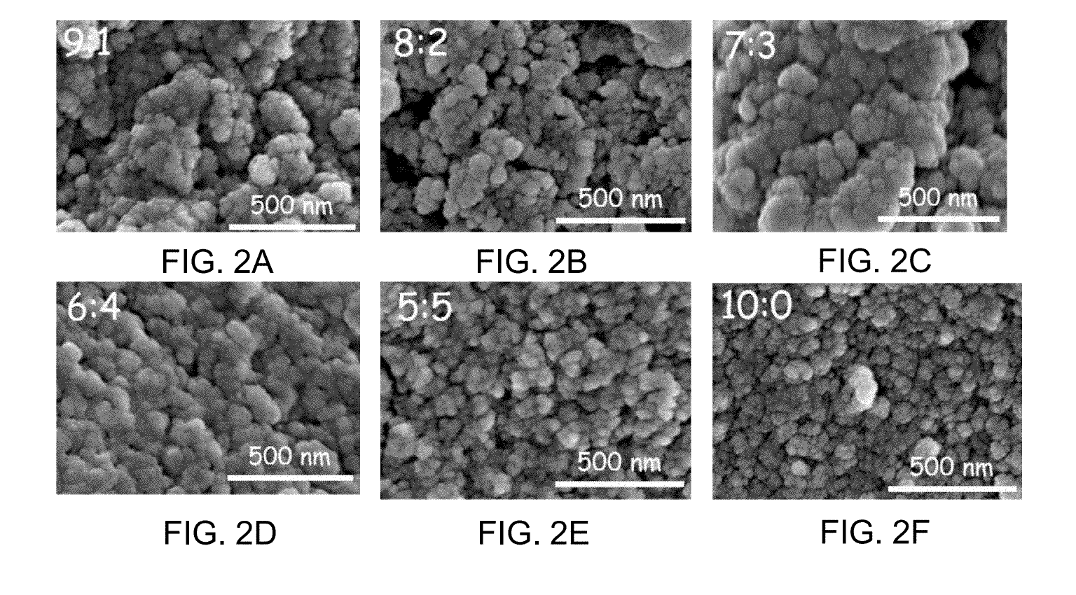

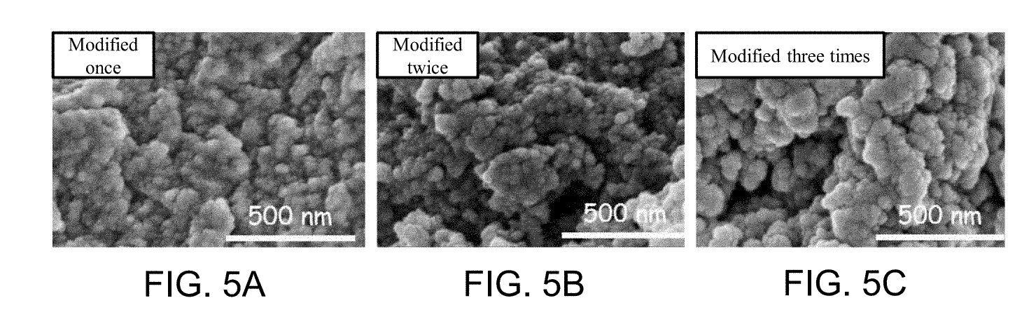

It can be known from FIG. 2A to FIG. 2F that, the Mg-doped alumina aerogel of experimental examples 1 to 5 and the alumina aerogel of comparative example 1 are all three-dimensional cross-linked network structures. Moreover, it can be known from FIG. 5A to FIG. 5C that, even if the Mg-doped alumina aerogel is modified by an amine group, a three-dimensional cross-linked network structure can still be maintained.

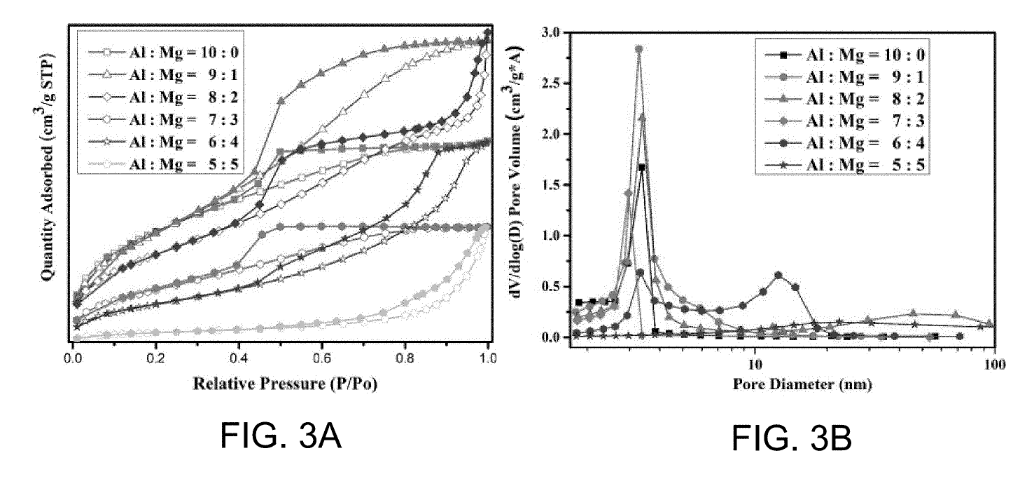

It can be known from the nitrogen adsorption and desorption curve of FIG. 3A that, comparative example 1 shows a type IV isotherm, which means that the alumina aerogel of comparative example 1 is a mesoporous material. As the magnesium amount is increased, hysteresis phenomenon tends to decrease, and the overall pore structure type tends to be microporous structures. Moreover, according to Table 1, when the amount reaches 7:3, the average pore diameter is about 2.74 nm, and when the amount reaches 6:4, the average pore diameter thereof shows microporous structures. It can be known from FIG. 3 that, when magnesium is added, the phenomenon of increased pore diameter occurs, but since the ion radii of magnesium and aluminum are similar, changes in pore characteristics do not occur. It can be known from Table 1 that, the specific surface areas of examples 1 to 3 are greater than those of experimental examples 4 and 5, indicating when the molar ratio of aluminum and magnesium is 7:3 to 8:2, the stability of the three-dimensional cross-linked network structure of the Mg-doped alumina aerogel can be maintained.

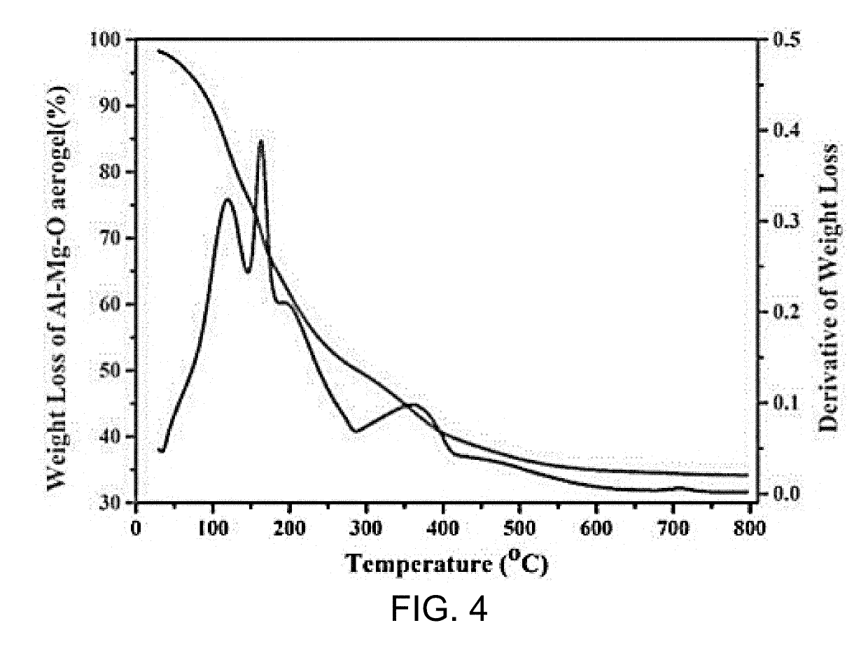

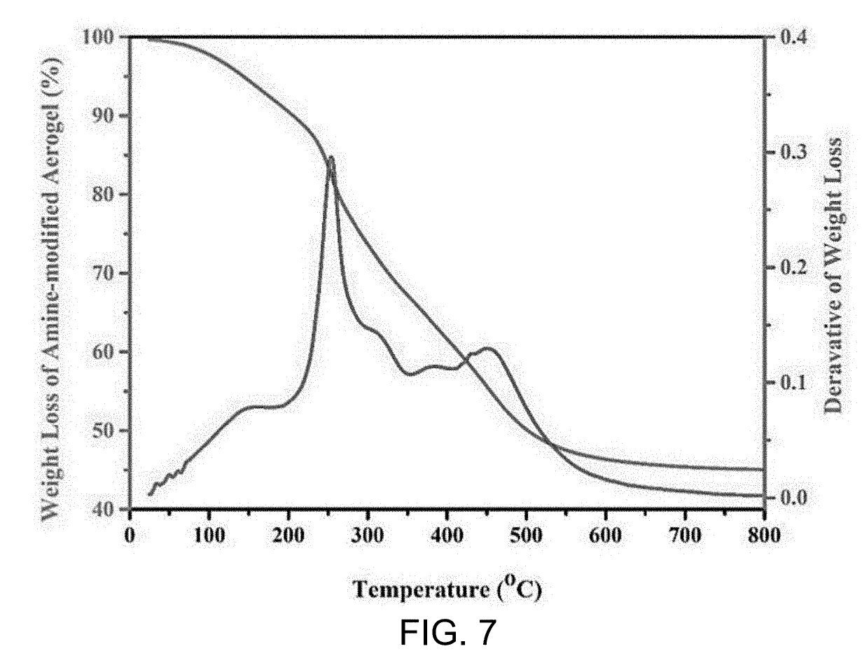

It can be known from FIG. 4 that, a large amount of heat loss occurs at 100° C. to 200° C. in example 2, with the reason being many hydroxyl groups are present in the Mg-doped alumina aerogel. In example 8, the Mg-doped alumina aerogel of example 2 is modified by an amine group. According to FIG. 7, the thermal stability of example 8 is better at 100° C. to 250° C., with the reason being hydroxyl groups are reduced by modifying amine groups on the three-dimensional cross-linked network structure.

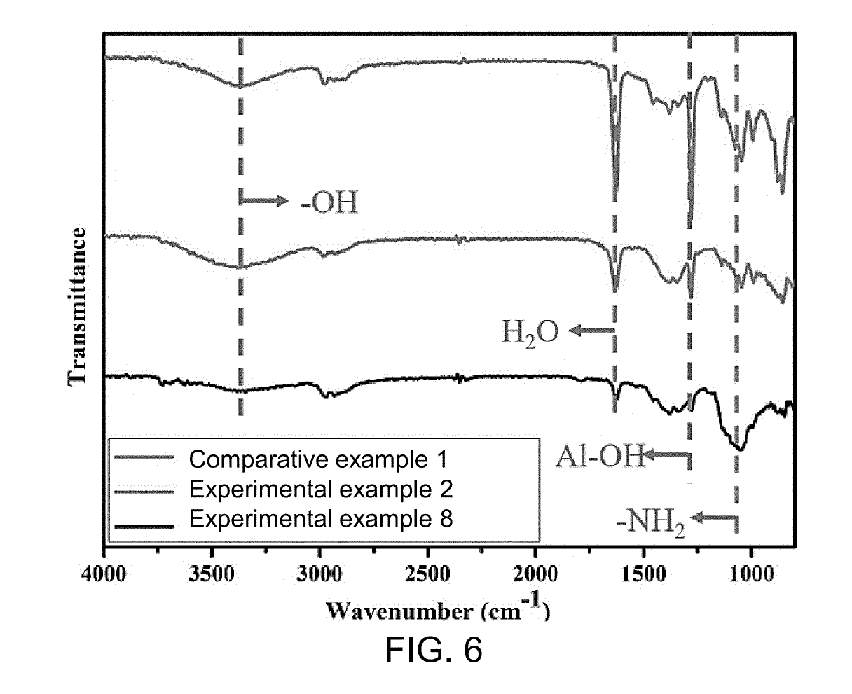

Moreover, according to FIG. 6, the characteristic peaks having wavelengths of 3450 cm⁻¹ (corresponding to hydroxyl group) and 1635 cm⁻¹ (corresponding to H₂O) of example 8 are significantly smaller than those of comparative example 1 and example 2, and a characteristic peak having a wavelength of 1150 cm⁻¹ (corresponding to —NH₂) occurs in example 8. This indicates a dehydration reaction occurs between 3-aminopropyl trimethoxysilane and the hydroxyl groups on the Mg-doped alumina aerogel surface (as shown in formula (V) above), and as a result a Mg-doped alumina aerogel modified by an amine group is obtained. Moreover, the characteristic peaks 620 cm⁻¹, 782 cm⁻¹, and 880 cm⁻¹ come from contributions of Al—O, and the characteristic peaks of examples 2 and 8 are significantly smaller than those of comparative example 1, indicating aluminum is replaced by magnesium, such that the characteristic peaks of 620 cm⁻¹, 782 cm⁻¹, and 880 cm⁻¹ are reduced.

Moreover, according to Table 1, when the molar ratio of aluminum and magnesium is 6:4 to 8:2, the Lewis acid and the Lewis base in the Mg-doped alumina aerogel reach a better balance. When the Lewis acid and the Lewis base reach better balance, the active site of the Lewis acid used with the epoxide can be sufficient, such that carbon dioxide is effectively reacted with propylene oxide, such that the conversion rate of propylene oxide is increased and the yield of propylene carbonate is better. Moreover, the active site of the Lewis base used with carbon dioxide can also be sufficient, such that the active epoxide is effectively reacted with carbon dioxide and is not self-polymerized to form a polypropylene byproduct, and therefore the selectivity of propylene carbonate is increased and the yield of propylene carbonate is better. Moreover, when the molar ratio of aluminum and magnesium is 8:2, the Lewis acid and the Lewis base in the Mg-doped alumina aerogel reach the optimal balance.

According to Table 2, the amine group of the Mg-doped alumina aerogel modified by an amine group is used as the active site of the Lewis base to increase the selectivity of propylene carbonate so as to increase the yield of propylene carbonate. When times of modifications is one to three times, the selectivity of propylene carbonate can be increased, and therefore the yield of propylene carbonate is increased. Moreover, according to experimental example 8 of Table 2, when amino group-modification was performed three times, the propylene oxide conversion and propylene carbonate selectivity reach 99% or more in the cycloaddition reaction of epoxide and carbon dioxide in the Mg-doped alumina aerogel modified by an amino group without the addition of a co-catalyst and a solvent. Therefore, the Mg-doped alumina aerogel modified by an amino group of experimental example 8 can be applied in mass production, and the cost thereof is lower than other known catalysts, and therefore has significant commercial potential.

Moreover, according to Table 2, in example 10, the alumina aerogel of comparative example 1 is modified by an amine group to obtain an alumina aerogel modified by an amine group. The propylene oxide conversion rate and the selectivity and yield of propylene carbonate of experimental example 10 are all higher than those of comparative example 1. Therefore, via the introduction of an amine group in the aerogel, catalytic efficiency can be enhanced. However, the catalytic efficiency of the Mg-doped alumina aerogel modified by an amine group of experimental example 8 is still better than that of the alumina aerogel modified by an amine group of experimental example 10.

Based on the above, the invention provides a Mg-doped alumina aerogel including a three-dimensional cross-linked network structure. The three-dimensional cross-linked network structure has a —Mg—O—Al— bond at least one the main chain, wherein the magnesium atom has Lewis base activity and the aluminum atom has Lewis acid activity, and therefore the Mg-doped alumina aerogel can be used as a catalyst. The Mg-doped alumina aerogel has the advantages of easy separation and environmental friendliness. Moreover, the invention further provides a Mg-doped alumina aerogel modified by an amine group. In addition to having the advantages of easy separation and environmental friendliness, the Mg-doped alumina aerogel also has better thermal stability, and since the amine group used as a Lewis base increases the selectivity of the catalytic reaction, better catalytic efficiency is achieved.

Although the invention has been described with reference to the above embodiments, it will be apparent to one of ordinary skill in the art that modifications to the described embodiments may be made without departing from the spirit of the invention. Accordingly, the scope of the invention is defined by the attached claims not by the above detailed descriptions.

What is claimed is:

1. A Mg-doped alumina aerogel used as a catalyst for preparing cyclic carbonate, the Mg-doped alumina aerogel comprising:
a three-dimensional cross-linked network structure comprising a plurality of magnesium atoms, a plurality of aluminum atoms, a plurality of oxygen atoms, and a plurality of hydrogen atoms,
the three-dimensional cross-linked network structure has a —Mg—O—Al— bond at least on the main chain.

2. The Mg-doped alumina aerogel of claim 1, wherein a molar ratio of the plurality of aluminum atoms and the plurality of magnesium atoms is 5:5 to 9:1.

3. The Mg-doped alumina aerogel of claim 1, wherein a molar ratio of the plurality of aluminum atoms and the plurality of magnesium atoms is 6:4 to 8:2.

4. The Mg-doped alumina aerogel of claim 1, wherein the three-dimensional cross-linked network structure further has an amine group.

5. A manufacturing method of a Mg-doped alumina aerogel, wherein the Mg-doped alumina aerogel is used as a catalyst for preparing cyclic carbonate, the manufacturing method of the Mg-doped alumina aerogel comprising:
performing a hydrolysis condensation reaction on an aluminum salt and a magnesium salt in the presence of an epoxide and a solvent to form a gel; and
performing a drying treatment on the gel to form the Mg-doped alumina aerogel.

6. The manufacturing method of the Mg-doped alumina aerogel of claim 5, further comprising an amine group modification step, in which a mixture of a silane compound containing an amine group and an alcohol solvent is added in the gel.

7. The manufacturing method of the Mg-doped alumina aerogel of claim 6, wherein the amine group modification step is performed one to three times.

8. The manufacturing method of the Mg-doped alumina aerogel of claim 5, wherein the aluminum salt is aluminum nitrate.

9. The manufacturing method of the Mg-doped alumina aerogel of claim 5, wherein the magnesium salt is magnesium nitrate.

10. The manufacturing method of the Mg-doped alumina aerogel of claim 5, wherein the drying treatment is a supercritical fluid drying method.
